(19) United States
(12) Patent Application Publication
(10) Pub. No.: US 2003/0149350 A1
(43) Pub. Date: Aug. 7, 2003

(54) Glaucoma screening system and method

(76) Inventor: Vittorio Porciatti, Miami, FL (US)

Correspondence Address: PAUL D. BIANCO, FLEIT, KAIN, GIBBONS, GUTMAN & BONGINI P.L., 601 BRICKELL KEY DRIVE, SUITE 404, MIAMI, FL, 33131, US

(21) Appl. No.: 10/357,080

(22) Filed: Feb. 3, 2003

Related U.S. Application Data
(60) Provisional application No. 60/354,805, filed on Feb. 5, 2002.

(51) Int. Cl. A61B 3/16

(52) U.S. Cl. 600/399

(57) ABSTRACT

A system, method and computer readable medium for efficiently screening patients for glaucoma is disclosed. The method includes attaching electrodes to a patient for measuring the response to stimulus. Then, a first set of stimuli patterns is presented on a display and a first set of signals from the electrodes is received. Then, a second set of stimuli patterns is presented on a display and a second set of signals from the electrodes is received. Next, the first set of signals is averaged with the second set of signals to produce a source data set. The source data set is compared with control data. Finally, using the information from the comparing step, it is determined whether the patient is afflicted with glaucoma.

### CROSS REFERENCE TO RELATED APPLICATION

The benefit of Provisional U.S. Patent Application No. 60/354,805, filed Feb. 5, 2002, is claimed under 35 U.S.C. § 119(e). The contents of this application are hereby incorporated by reference.

### FIELD OF THE INVENTION

This invention generally relates to the field of glaucoma screening and more specifically to glaucoma screening using a pattern electroretinogram.

### BACKGROUND OF THE INVENTION

Glaucoma is a specific pattern of optic nerve damage and visual field loss. In the year 2000, it was estimated that three million Americans and sixty-seven million people worldwide have glaucoma. At least half of the people afflicted with glaucoma are unaware of this because glaucoma is usually asymptomatic. When untreated, glaucoma is a leading cause of blindness. Risk factors for glaucoma are:

People over the age of 45

People who have a family history of glaucoma

People with abnormally high intraocular pressure (IOP)

People of African descent

People who have diabetes, myopia, a previous eye injury, or previous steroid/cortisone use

Ophthalmologists are often faced with patients suspected of having glaucoma because of increased cupping of the optic disk, associated with other signs (such as increased IOP, disk hemorrhages, disk notching and disk asymmetry) or other risk factors stated above. The typical approach to patients suspected of having glaucoma is to take a picture of the optic disk and check for defects of the Humphrey visual field (HVF). This approach represents the gold standard for determining whether or not a patient has glaucoma. When the HVF is normal, the question is whether the patient may have glaucoma in the early stages, which the HVF is incapable of detecting.

Indeed, previous evidence in the retinas of human donors with a history of glaucoma, and experiments involving monkeys with experimental glaucoma has indicated that the first significant HVF (either white-on-white, blue-on-yellow, or red-on-green) defects appear when at least 30-50% of ganglion cells have degenerated. Further loss of ganglion cells results in an exponential deterioration of the HVF.

Another necessary step in determining whether a patient suspected of having glaucoma is afflicted with the disease is to ask the patient to return every six months to check for initial defects of the HVF. One should take into account that the HVF is based on the detection of the lowest perceived luminance of tiny spots of lights presented randomly in different parts of a screen placed in front of a patient. Therefore, the HVF has an intrinsic difficulty and variability. In addition, being a subjective response, the HVF depends on several psychological variables such as learning, fatigue, and attention level. These drawbacks are of greater impact in the aged, where there is a larger prevalence of glaucoma. Due to the intrinsic fluctuation of the patients' performance, it may take several sessions, or even years, to establish whether an initial defect of the visual field is consistent. This causes growing frustration in the patients, who are anxious to know whether or not they have glaucoma and whether or not they have to be treated.

The pattern electroretinogram (PERG) is a special kind of electroretinogram whereby the patient observes a pattern of alternating dark and white bars or checks presented on a monitor, while the electrical response of the eye is recorded. As opposed to the ordinary flash-ERG, the PERG is selectively altered when retinal ganglion cells are in dysfunction, including glaucoma in the early stage. The PERG is reported to be altered in many patients with increased IOP but normal visual fields. In addition, an abnormal PERG may predict future impairment of the visual field. Therefore, the PERG may represent a useful tool for the early detection and follow up of glaucoma suspects. In the past, however, the PERG has been underused as a screening test for glaucoma. Present PERG techniques do not appear patient-friendly or user-friendly, since they typically require topical anesthesia, uncomfortable and unstable corneal electrodes, and interpretation of the response waveform.

The pattern electroretinogram, however, represents the only technique available to specifically, directly, and objectively evaluate the function of retinal ganglion cells in humans. The PERG has been shown to be dramatically altered in all patients with glaucoma and significantly altered in a large population (over 50%) of subjects with ocular hypertension but with normal visual field. An abnormal PERG in glaucoma suspects may predict future impairment of the visual field. In addition, in patients with ocular hypertension, the PERG may signal beneficial effects of treatment and disclose abnormal recovery of ganglion cells function after transient increase of intraocular pressure. Moreover, comparative evaluation of techniques for glaucoma detection shows that the PERG has a better sensitivity and specificity over computer-assisted perimetry. Thus, the PERG may serve as a more efficient technique for the earlier diagnosis and monitoring of early stages of glaucoma, particularly since it is an objective response.

In the past, however, the PERG has been virtually ignored as a screening test for glaucoma. As explained above, present clinical standards for the PERG are not patient-friendly or user-friendly. Additionally, the PERG is a response of much smaller amplitude as compared to the ordinary flash-ERG (by a factor of 100 or more) and more difficult to record. In order to obtain responses with the highest possible amplitude, the recording electrode is typically placed on the cornea. This usually requires topical anesthesia. Responses with highest amplitude are obtained when corneal electrodes are embedded in a contact lens or in a speculum. With these electrodes, the visibility of the pattern stimulus results are somehow impaired. Therefore, the refraction of the eye and the best-corrected visual acuity for the viewing distance must be re-evaluated with ERG electrodes in place. This requires time, causes discomfort for the patients, and requires the presence of a skilled operator. Further, this procedure has to be repeated at any successive recording session.

To avoid interference with vision, corneal electrodes consisting of tiny threads of carbon fibers or a tiny lamina of gold may be inserted into the conjunctival fornix or leaned on the corneal surface. For brief recording sessions, some patients may accept these corneal electrodes without topical anesthesia. However, the stability of these electrodes is poor, since they can change location, or even be expelled, from the eye after each blink. The use of these electrodes requires a skilled operator, able to insert the electrode in the same location and check for possible changes during the recording session. In addition, the use of these electrodes may be not extended to all patients, in particular the older patients. Extended recording for testing the effect of pharmacological treatment may cause corneal damage to patients.

Therefore, a need exists for an improved system and method to efficiently screen patients for glaucoma.

### SUMMARY OF THE INVENTION

Briefly, in accordance with the present invention, disclosed is a system, method and computer readable medium for efficiently screening patients for glaucoma. In an embodiment of the present invention, the method on a computer system includes attaching a plurality of electrodes to the skin on the head of a patient. Then, a first set of stimuli patterns are presented on a display viewed by the patient. In response to the first set of stimuli patterns, a first set of signals from the plurality of electrodes is received. Then, a second set of stimuli patterns are presented on a display viewed by the patient. In response to the second set of stimuli patterns, a second set of signals from the plurality of electrodes is received. Next, the first set of signals is averaged with the second set of signals to produce a source data set. The source data set is compared with control data. Finally, using the information from the comparing step it is determined whether the patient is afflicted with glaucoma.

In an embodiment of the present invention, the method includes further processing of the first set of signals and the second set of signals received from the plurality of electrodes. First, a set of signals is amplified. Then, the set of signals is filtered and digitized. Subsequently, the set of signals is averaged to produce an averaged set of signals. The averaged set of signals is then noise cancelled.

In another embodiment of the present invention, the control data to which the source data set is compared comprises age-matched normal data. Further, a result of the comparing step is a resultant value represented in standard deviations from an average of the control data.

The described embodiments of the present invention are advantageous as they allow for the quick and efficient screening of a patient for glaucoma. The proposed method requires only a few minutes and a trained technician (as opposed to a physician or a highly skilled technician) to complete a screening of a patient for glaucoma. Further, the proposed method results in an immediate rendering of a diagnosis. This is beneficial as it reduces the amount of time and resources necessary to render a diagnosis.

Another advantage of the present invention is that the method of the present invention can use non-invasive skin electrodes instead of corneal electrodes, which cause discomfort, require larger periods of time and require a skilled technician. The proposed method radically eliminates the problem of optical degradation of the pattern stimulus, the problem of electrode stability, and the problem of dependency on a skilled operator.

An obvious drawback of non-corneal electrodes, as compared to corneal electrodes, is the reduction of signal amplitude (by a factor of 2-3). However, responses with a signal-to-noise ratio (S/N) of ten or more may be easily obtained if the characteristics of the stimulus are chosen to obtain responses of maximal amplitude, the averaging is adequate to reduce the electrical activity uncorrelated with contrast-alternating pattern, and the signal processing is performed in the frequency domain to isolate the signal (response component at the frequency of contrast-reversal) from the noise (at frequencies different from that of contrast-reversal).

The inventor has previously shown that a PERG can be reliably recorded by means of skin electrodes. The properties of such a PERG are comparable to those obtained with corneal electrodes. In particular, the non-invasive PERG changes with age, and is altered in many patients with ocular hypertension despite normal visual field. In addition, the signal-to-noise ratio of the non-invasive PERG is adequate to show the activity originating from different subpopulations of ganglion cells when the chromatic characteristics of the stimulus are changed.

Yet another advantage of the present invention is that the proposed method provides sensitivity in detecting early glaucomatous dysfunction of retinal ganglion cells. This is beneficial as it provides the ability to detect initial dysfunction of ganglion cells in glaucoma patients before the occurrence of significant defects in the automated standard perimetry. Further, the proposed method allows for extended recording of a PERG, which allows for monitoring the progression of a PERG and evaluating the effects of pharmacological treatment.

The foregoing and other features and advantages of the present invention will be apparent from the following more particular description of the preferred embodiments of the invention, as illustrated in the accompanying drawings.

### BRIEF DESCRIPTION OF THE DRAWINGS

The subject matter, which is regarded as the invention, is particularly pointed out and distinctly claimed in the claims at the conclusion of the specification. The foregoing and other features and also the advantages of the invention will be apparent from the following detailed description taken in conjunction with the accompanying drawings. Additionally, the left-most digit of a reference number identifies the drawing in which the reference number first appears.

FIG. 1 is a block diagram illustrating the overall system architecture of one embodiment of the present invention.

FIG. 2 is a representation of age-dependent PERG changes in amplitude.

FIG. 3 is a representation of age-dependent PERG changes in phase.

FIG. 4 is a representation of amplitude interocular PERG asymmetry.

FIG. 5 is a representation of phase interocular PERG asymmetry.

FIG. 6 is a representation of a normal PERG waveform, according to the present invention.

FIG. 7 is a representation of a normal PERG value compared to average control data, according to the present invention.

FIG. 8 is a representation of an abnormal PERG waveform, according to the present invention.

FIG. 9 is a representation of an abnormal PERG value compared to average control data, according to the present invention.

FIG. 10 is a block diagram of a computer system useful for implementing the present invention.

### DESCRIPTION OF THE PREFERRED EMBODIMENTS

Overview

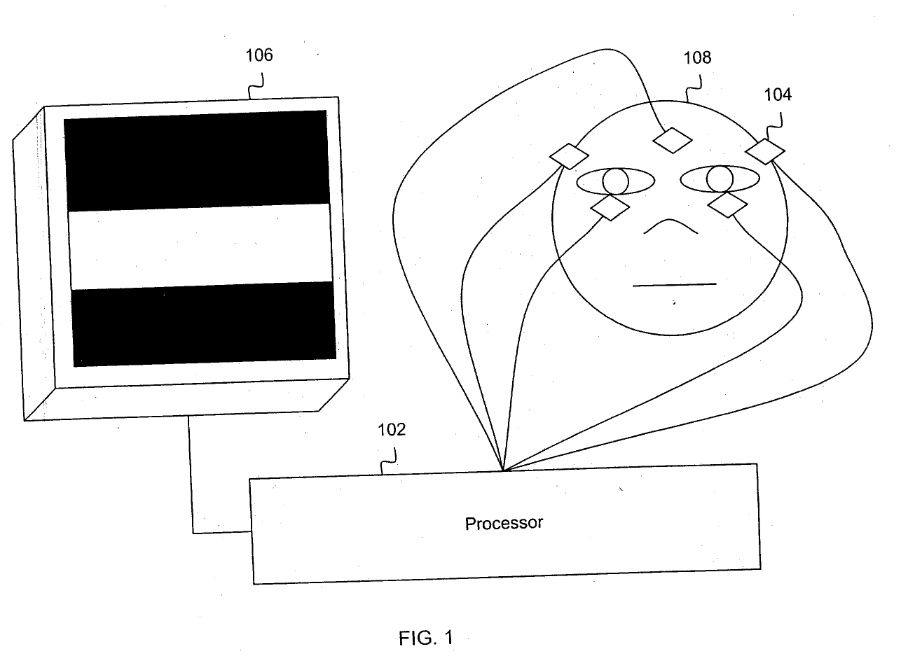

FIG. 1 is a block diagram illustrating the overall system architecture of one embodiment of the present invention. This figure shows a patient 108 undergoing a pattern electroretinogram (PERG). Patient 108 views a display 106 presenting pattern stimuli, wherein the display 106 is controlled by a processor 102. A group of electrodes 104 are connected to patient 108. As shown, electrodes 104 are in contact with skin on the head of patient 108, for measuring the ophthalmic reactions of patient 108 to the pattern stimuli. Alternatively, electrodes 104 could be corneal electrodes. Regardless of their number or placement, signals from the electrodes 104 are gathered by processor 102. The processor 102 represents a computer system for controlling display 106, interacting with electrodes 104 and analyzing signals from electrodes 104.

The computer system of processor 102 comprises one or more Personal Computers (PCs) (e.g., IBM or compatible PC workstations running the Microsoft Windows 95/98/2000/ME/CE/NT/XP operating system, Macintosh computers running the Mac OS operating system, or equivalent), Personal Digital Assistants (PDAs), game consoles or any other computer processing devices. In an embodiment of the present invention, the computer system of processor 102 comprises one or more server systems (e.g., SUN Ultra workstations running the SunOS or AIX operating system or IBM RS/6000 workstations and servers running the AIX operating system). The display 106 is any commercially available NTSC display, PAL display, HDTV display, progressive scan display, or the like.

In an alternate embodiment of the present invention, FIG. 1 includes a network to which the computer system of processor 102 is connected. The network is a circuit switched network, such as the Public Service Telephone Network (PSTN). In another embodiment of the present invention, the network is a packet switched network. The packet switched network is a wide area network (WAN), such as the global Internet, a private WAN, a local area network (LAN), a telecommunications network or any combination of the above-mentioned networks. The network is a wired network, a wireless network, a broadcast network or a point-to-point network.

Operation of the Invention

The following section describes the overall process of screening a patient for glaucoma, according to one embodiment of the present invention. In a first step, the patient is prepared for the PERG. Next, the PERG is administered. Then, the results of the PERG are analyzed. Finally, the diagnosis is rendered. The patient preparation process, the PERG administration process, the analysis process and the diagnosis process are explained in greater detail below.

In an optional step before the PERG administration process, set-up information is entered into processor 102. In this step, a technician or the physician administering the PERG enters information that is used in the analysis process and/or the diagnosis process. Set-up information can include the age of the patient, the gender of the patient, the previous medical history of the patient, the results of previous PERGs on the patient, the health status of the patient and any information relating to the ophthalmic status of the patient.

Patient Preparation

The following section describes the patient preparation process, according to one embodiment of the present invention. In a first step, the skin of patient 108 is cleaned in the areas in which the electrodes 104 are to be attached. The area cleaned is about a square centimeter in size for each electrode 104. This reduces skin impedance below 5,000 Ohms and increases conductivity between the electrode and the skin. This step takes about one minute.

Then, the electrodes 104 are filled with conductive jelly to increase conductivity between the electrode and the skin. Next, the electrodes 104 are attached to the skin in the areas previously cleaned. Preferably, surgical tape (1.5×1.5 cm or any size appropriate to the size of the electrodes) is used to attach the electrodes 104 to the skin.

In an embodiment of the present invention, five electrodes 104 are used. Preferably, the electrodes 104 are placed one on the lower eyelid of the left eye, one on the lower eyelid of the right eye, one on the left temple, one on the right temple and one on the center of the forehead. See FIG. 1 for an example of the placement of the five electrodes 104 on the skin of the head of the patient 108. For lower eyelid electrodes, the upper margin of the electrodes is kept about 0.5 mm below eyelashes. The central forehead electrode is placed approximately 3 cm above the bridge of the nose, while the temporal electrodes are placed about 3 cm lateral to the lateral cantus. This step takes about 0.5 minutes.

Next, the electrodes 104 are plugged into processor 102. The patient 108 must be kept electrically isolated from the ground in order to prevent signals entering the electrodes 104 from grounding out. This step takes about 0.5 minutes.

Subsequently, the patient 108 places his chin on a chin rest placed in front of display 106. In such a position, the patient 108 can view display 106 with little movement of his head. Preferably, the distance between the eye of patient 108 and the display 106 is about 30 cm. This step takes a few seconds.

Finally, the patient 108 focuses his vision on a fixation spot located at the center of the display 106. When necessary, optical correction is given to the patient 108 for the viewing distance. Fixation time lasts about 1.5 minutes, during which the patient 108 is allowed to blink freely.

PERG Administration

The following section describes the PERG administration process, according to one embodiment of the present invention. In a first step, a first set of stimuli patterns are presented on the display 106. In an exemplary embodiment, the stimuli patterns consist of a black-white bar grating with a square-wave profile. The bars alternate in contrast without changes in mean luminance.

In an embodiment of the present invention, the stimuli patterns are displayed on display 106 and masked to a circular field of about 14.7 cm diameter. When patient 108 looks at the center of display 106 from about 30 cm viewing distance, the stimuli patterns cover a retinal area of about 24 degree diameter centered on the fovea. The mean luminance of the display is 40 candela/square meter, the spatial frequency of the grating is 1.6 cycles/degree, and the contrast of the grating is 98 percent. The contrast is defined as the difference in luminance between light and dark bars, divided by the sum of their luminances. The bars alternate in counterphase at 8.14 Hz (temporal period 122.8 ms, two contrast reversals per period) without changes in mean luminance.

Then, a first data set is received from the electrodes 104. Preferably, electrical signals from the electrodes 104 are fed into a two-channel differential amplifier located at the processor 102.

Next, the first data set is processed. In this step, the electrical signals from the electrodes 104 are fed into a two-channel differential amplifier which amplifies, filters and digitizes the electrical signals. The electrode impedance is automatically evaluated by the amplifier. The processed first data set is stored by processor 102.

In an embodiment of the present invention, the two-channel differential amplifier amplifies the electrical signals 100,000 fold, filters the electrical signals at 1-30 Hz, and digitizes the electrical signals with 12 bit resolution at 4.169 Hz. The electrode impedance is automatically evaluated by the amplifier, and an LED light indicates whether the impedance is acceptable or higher than 5,000 Ohm. The LED allows the person administering the PERG to adjust the electrodes 104 if the impedance is not within acceptable limits.

Preferably, the PERG waveform is obtained by averaging 600 sweeps of 122.8 ms duration, in synchrony with the stimulus alternation in display 106. Two independent response blocks of 330 sweeps each are recorded. For each block, the first 30 sweeps are rejected from the average to eliminate the spurious effect of the stimulus onset, and to allow a steady-state condition of recording. Sweeps containing spurious signals originating from eye blinking or gross eye movements are automatically rejected, and additional sweeps are averaged. Typically, 5 to 20 spurious sweeps per recording are rejected. The amount of rejections has no effect on the averaged waveform, although the recording time is somewhat longer when the rejection rate is higher.

Further, a “noise” waveform can be obtained simultaneously with the response waveform by subtracting even and odd sweeps. The PERG and noise waveforms can then be automatically submitted to a Digital Fourier Transform to isolate the harmonic component at the contrast-reversal frequency (16.3 Hz), and the amplitude (in μV) and phase (in π rad) are computed. In normal subjects, the PERG-to-noise amplitude ratio is generally 10.0 or higher with an average ratio of about 13.7.

In order to assess response consistency, multiple PERG's can be recorded. For example, the previous steps can be repeated for another data set. Thus, a second set of stimuli patterns are presented on the display 106 in a manner identical to the pattern presentation step above. Then, a second data set is received from the electrodes 104 in a manner identical to the data set reception step above. Lastly, the second data set is processed in a manner identical to the data set processing step above. The processed second data set is stored by processor 102.

PERG Comparison

The following section describes the PERG comparison process, according to one embodiment of the present invention. In a first step, the first data set and the second data set are compared and averaged to produce a source data set. Specifically, the first data set and the second data set are compared to have a measure of response consistency (Coefficient of Variation (CV):SD/mean) and then averaged. Typically, the CV is of the order of 5-10 percent. Occasionally, the CV may be higher. In such cases, one or two additional data sets can be recorded to reduce the standard deviation and keep the CV below 20 percent.

Next, the source data set is analyzed and compared to control data. The amplitude and phase of the source data set are compared with a database of age-matched normal controls, and then expressed in Standard Deviations (SDs) from the normal average. Within two SDs from the normal average, the PERG amplitude and phase are considered normal.

In one embodiment of the present invention, normative data have been obtained from a population of healthy subjects (n=92) aged 22-85 years with no ocular or systemic disease, with Snellen visual acuity of 20/20 or higher, and with normal intraocular pressure and Standard Humphrey automated perimetry. In these subjects, the amplitude and phase decrease progressively with age. Amplitude data are well fitted with a linear regression line on log-log plot. Since the residuals are independent of age, all data can be collapsed in a single statistical group. The residuals have a normal distribution with a SD of ±1.0 dB. The phase data are also well fitted with a linear regression line (on a linear-linear plot). As for the amplitude data, the residuals are independent of age, and can be collapsed in a single statistical group. The phase residuals have a normal distribution with a SD of ±0.1 π rad.

In another embodiment of the present invention, normative data (n=26) have been obtained for test-retest variability (PERG obtained by the same operator on the same subject in different days), and normative data (n=8) have also been obtained for operator-dependent variability (PERG obtained by different operators on the same subject in different days). The test-retest Coefficients of Variation for amplitude and phase are 8.2±5.0 and 1.7±1.4, respectively, while the operator-dependent Coefficients of Variation are similar to the test-retest variability. In some control subjects (n=6) the electrodes have been kept in place for about 8 hours, and the PERG recorded twice to assess the variability during an extended recording. Changes in amplitude and phase never exceeded the range of normal test-retest variability.

In yet another embodiment of the present invention, to determine the deviation from normal in patients, the amplitude and phase of the source data set are subtracted from those predicted from the equations of regression lines with age of the normal population. The deviation from the predicted value is computed by dividing the resulting difference by the SD of amplitude and phase of control subjects.

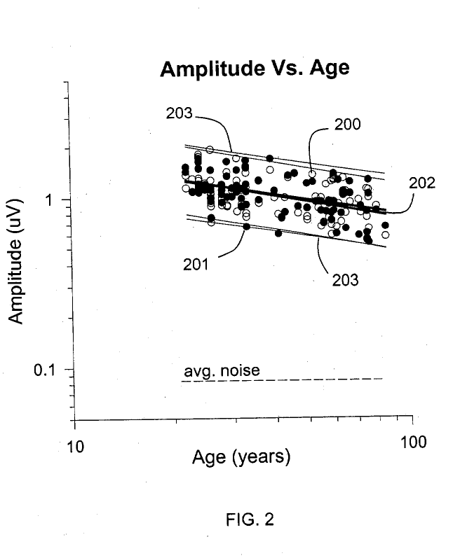

FIG. 2 represents age-dependent PERG changes. This figure is a scatterplot of amplitude of the PERG recorded from both eyes of 92 subjects ranging from 22 to 85 years of age. Open symbols 200 represent right eyes, and closed symbols 201 represent left eyes. Thick lines 202 represent the regression lines, and thin lines 203 represent the 95 percent confidence intervals. The amplitude data could be best fitted with a linear regression line on log-log coordinates.

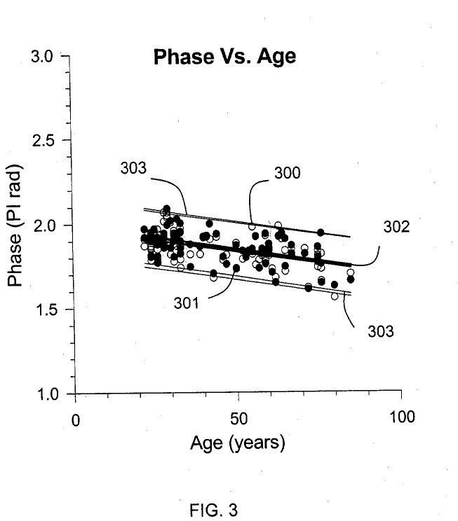

FIG. 3 also represents age-dependent PERG changes. This figure is a scatterplot of phase of the PERG recorded from both eyes of 92 subjects ranging from 22 to 85 years of age. Open symbols 300 represent right eyes, and closed symbols 301 represent left eyes. Thick lines 302 represent the regression lines, and thin lines 303 represent the 95 percent confidence intervals. The phase data could be best fitted with a linear regression line on linear-linear coordinates.

In normal controls, the PERG amplitude and phase decrease (the latency increases) with increasing age. Age-related PERG changes result in part from reduction of retinal illuminance due to senile miosis. When the response is normalized for senile miosis, however, the PERG of aged subjects is still substantially reduced and delayed as compared to the PERG of younger subjects. This indicates, at least in part, an age-dependent loss of retinal ganglion cells.

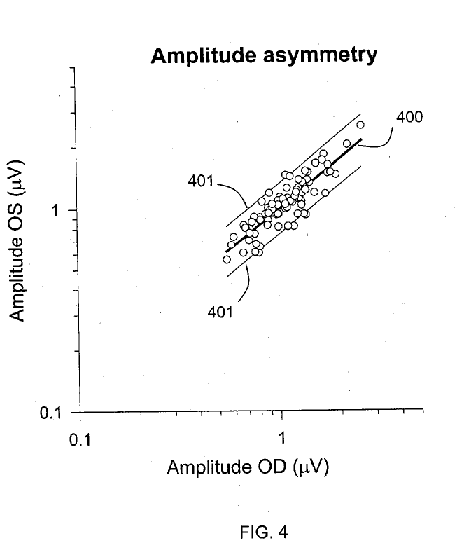

FIG. 4 represents interocular PERG asymmetry. Amplitude data of the left eyes are plotted against corresponding data of the right eyes. This figure shows the regression line 400 as well as the 95 percent confidence intervals 401.

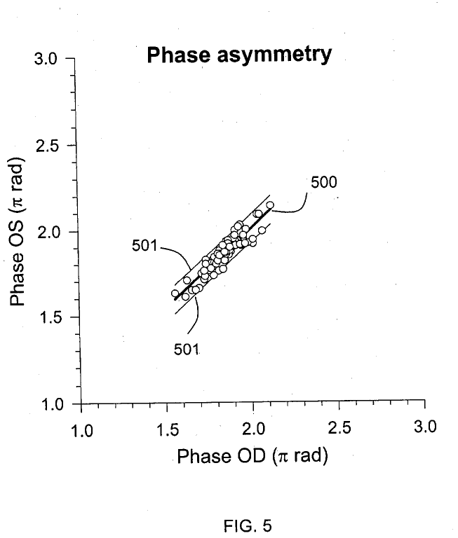

FIG. 5 also represents interocular PERG asymmetry. Phase data of the left eyes are plotted against corresponding data of the right eyes. This figure shows the regression line 500 as well as the 95 percent confidence intervals 501.

Interocular asymmetry may offer additional information about early retinal ganglion cell dysfunction. For example, if a disease progresses asymmetrically as with glaucoma, then the amount of asymmetry may be expected to be abnormally high even before the PERG amplitude and phase exceed the 95 percent confidence limits of normal controls.

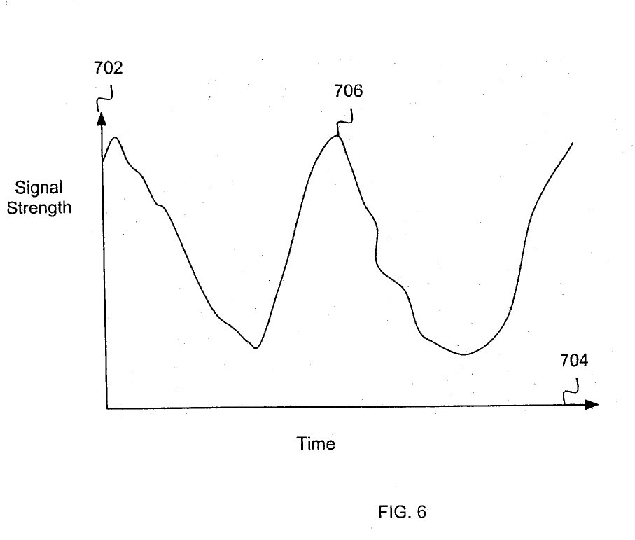

FIG. 6 is a representation of a normal PERG waveform, according to the present invention. This figure shows a normal PERG waveform 706 (representing the source data set) against a signal strength 702 versus time 704 coordinate system.

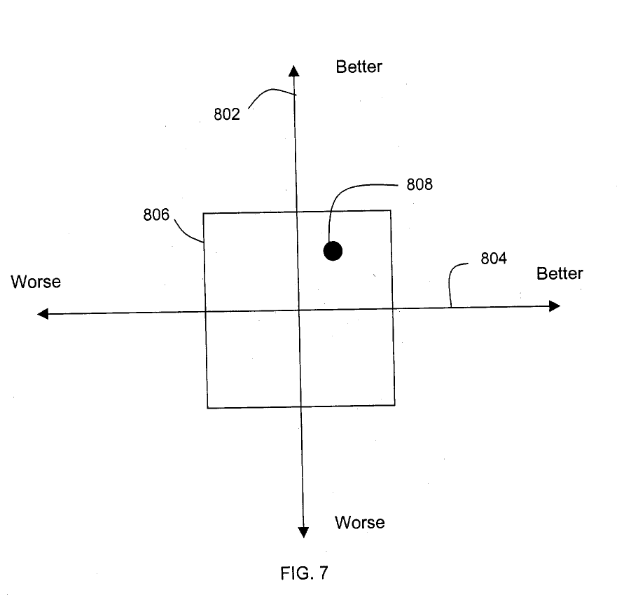

FIG. 7 is a representation of a normal PERG value compared to average control data, according to the present invention. This figure shows a normal PERG value (representing the source data set) against a better versus worse coordinate system. The x-axis 804 represents a better, or more normal, PERG value as it extends toward the positive range (towards the right) and a worse, or abnormal, PERG value as it extends toward the negative range (towards the left). The y-axis 802 represents a better, or more normal, PERG value as it extends toward the positive range (upwards) and a worse, or abnormal, PERG value as it extends toward the negative range (downwards). The loci 806 represents the predicted range of a normal PERG value. The point 808 represents the source data set located within the predicted range of a normal PERG value—within loci 806.

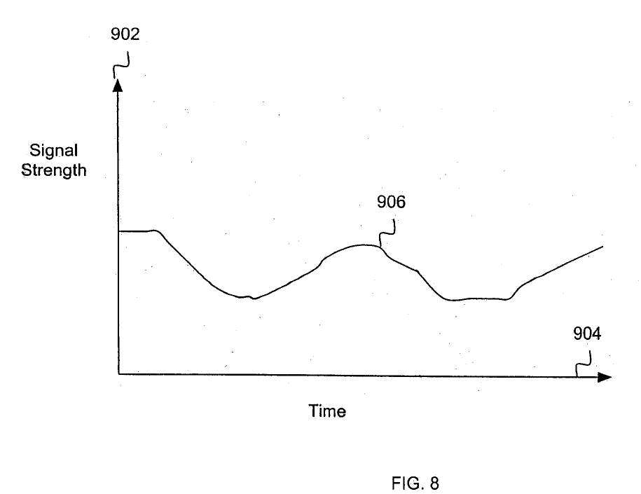

FIG. 8 is a representation of an abnormal PERG waveform, according to the present invention. This figure shows an abnormal PERG waveform 906 (representing the source data set) against a signal strength 902 versus time 904 coordinate system.

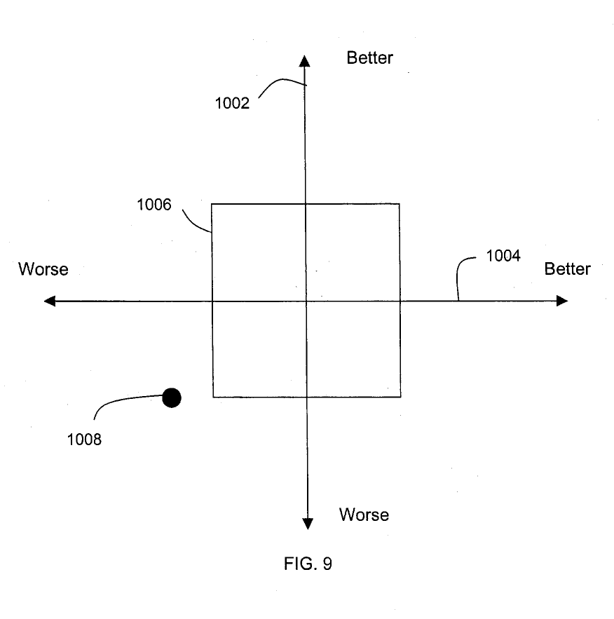

FIG. 9 is a representation of an abnormal PERG value compared to average control data, according to the present invention. This figure shows an abnormal PERG value (representing the source data set) against a better versus worse coordinate system. The x-axis 1004 represents a better, or more normal, PERG value as it extends toward the positive range (towards the right) and a worse, or abnormal, PERG value as it extends toward the negative range (towards the left). The y-axis 1002 represents a better, or more normal, PERG value as it extends toward the positive range (upwards) and a worse, or abnormal, PERG value as it extends toward the negative range (downwards). The loci 1006 represents the predicted range of a normal PERG value. The point 1008 represents the source data set located outside the predicted range of a normal PERG value—outside loci 1006. This indicates that the source data set represents an abnormal PERG value.

Diagnosis

The following section describes the diagnosis process, according to one embodiment of the present invention. In a first step, a physician or technician renders a diagnosis based upon the PERG comparison process described above. As shown in the figures and described in the above discussion, the results are easily interpreted based on the graphic representation of the data compared to controls. The diagnosis may be conclusive or inconclusive as to the presence of glaucoma.

Next, it is determined whether the diagnosis is conclusive. If the diagnosis is conclusive and the diagnosis is negative (glaucoma is present), then the patient 108 is directed to treatment for the glaucoma diagnosis. A negative diagnosis would result from an abnormal PERG waveform, as shown in FIG. 4 above, and an abnormal PERG value compared to average control data, as shown in FIG. 5 above. If the diagnosis is not conclusive, a follow up PERG is scheduled.

Exemplary Implementations

The present invention can be realized in hardware, software, or a combination of hardware and software. A system according to a preferred embodiment of the present invention can be realized in a centralized fashion in one computer system, or in a distributed fashion where different elements are spread across several interconnected computer systems. Any kind of computer system—or other apparatus adapted for carrying out the methods described herein—is suited. A typical combination of hardware and software could be a general-purpose computer system with a computer program that, when being loaded and executed, controls the computer system such that it carries out the methods described herein.

An embodiment of the present invention can also be embedded in a computer program product, which comprises all the features enabling the implementation of the methods described herein, and which—when loaded in a computer system—is able to carry out these methods. Computer program means or computer program in the present context mean any expression, in any language, code or notation, of a set of instructions intended to cause a system having an information processing capability to perform a particular function either directly or after either or both of the following a) conversion to another language, code or, notation; and b) reproduction in a different material form.

A computer system may include, inter alia, one or more computers and at least a computer readable medium, allowing a computer system, to read data, instructions, messages or message packets, and other computer readable information from the computer readable medium. The computer readable medium may include non-volatile memory, such as ROM, Flash memory, disk drive memory, CD-ROM, and other permanent storage. Additionally, a computer readable medium may include, for example, volatile storage such as RAM, buffers, cache memory, and network circuits. Furthermore, the computer readable medium may comprise computer readable information in a transitory state medium such as a network link and/or a network interface, including a wired network or a wireless network, that allow a computer system to read such computer readable information.

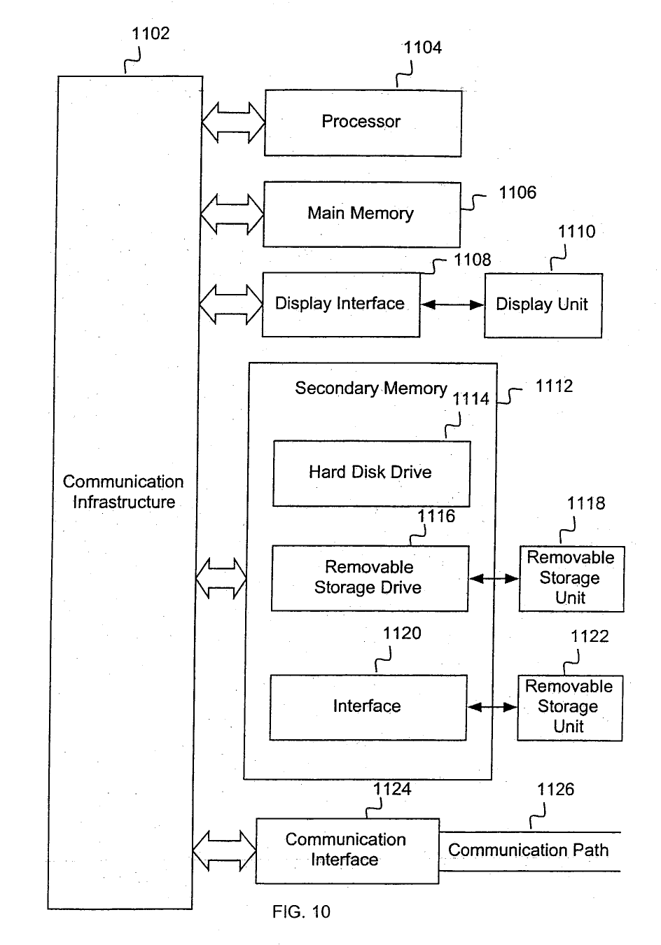

FIG. 10 is a block diagram of a computer system useful for implementing an embodiment of the present invention. The computer system includes one or more processors, such as processor 1104. The processor 1104 is connected to a communication infrastructure 1102 (e.g., a communications bus, cross-over bar, or network). Various software embodiments are described in terms of this exemplary computer system. After reading this description, it will become apparent to a person of ordinary skill in the relevant art(s) how to implement the invention using other computer systems and/or computer architectures.

The computer system can include a display interface 1108 that forwards graphics, text, and other data from the communication infrastructure 1102 (or from a frame buffer not shown) for display on the display unit 1110. The computer system also includes a main memory 1106, preferably random access memory (RAM), and may also include a secondary memory 1112. The secondary memory 1112 may include, for example, a hard disk drive 1114 and/or a removable storage drive 1116, representing a floppy disk drive, a magnetic tape drive, an optical disk drive, etc. The removable storage drive 1116 reads from and/or writes to a removable storage unit 1118 in a manner well known to those having ordinary skill in the art. Removable storage unit 1118, represents a floppy disk, magnetic tape, optical disk, etc. which is read by and written to by removable storage drive 1116. As will be appreciated, the removable storage unit 1118 includes a computer usable storage medium having stored therein computer software and/or data.

In alternative embodiments, the secondary memory 1112 may include other similar means for allowing computer programs or other instructions to be loaded into the computer system. Such means may include, for example, a removable storage unit 1122 and an interface 1120. Examples of such may include a program cartridge and cartridge interface (such as that found in video game devices), a removable memory chip (such as an EPROM, or PROM) and associated socket, and other removable storage units 1122 and interfaces 1120 which allow software and data to be transferred from the removable storage unit 1122 to the computer system.

The computer system may also include a communications interface 1124. Communications interface 1124 allows software and data to be transferred between the computer system and external devices. Examples of communications interface 1124 may include a modem, a network interface (such as an Ethernet card), a communications port, a PCMCIA slot and card, etc. Software and data transferred via communications interface 1124 are in the form of signals which may be, for example, electronic, electromagnetic, optical, or other signals capable of being received by communications interface 1124. These signals are provided to communications interface 1124 via a communications path (i.e., channel) 1126. This channel 1126 carries signals and may be implemented using wire or cable, fiber optics, a phone line, a cellular phone link, an RF link, and/or other communications channels.

In this document, the terms “computer program medium,” “computer usable medium,” and “computer readable medium” are used to generally refer to media such as main memory 1106 and secondary memory 1112, removable storage drive 1116, a hard disk installed in hard disk drive 1114, and signals. These computer program products are means for providing software to the computer system. The computer readable medium allows the computer system to read data, instructions, messages or message packets, and other computer readable information from the computer readable medium. The computer readable medium, for example, may include non-volatile memory, such as floppy, ROM, flash memory, disk drive memory, CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network, that allow a computer to read such computer readable information.

Computer programs (also called computer control logic) are stored in main memory 1106 and/or secondary memory 1112. Computer programs may also be received via communications interface 1124. Such computer programs, when executed, enable the computer system to perform the features of the present invention as discussed herein. In particular, the computer programs, when executed, enable the processor 1104 to perform the features of the computer system. Accordingly, such computer programs represent controllers of the computer system.

Conclusion

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

What is claimed is:

1. A glaucoma screening method for detecting glaucoma in a patient, the method comprising:
attaching a plurality of electrodes to the patient;
displaying a first set of stimuli patterns to the patient;
receiving, in response to the first set of stimuli patterns, a first set of signals from the plurality of electrodes;
processing the first set of signals to produce a source data set;
comparing the source data set with control data; and
determining whether the patient is afflicted with glaucoma based on the comparing step.

2. The method of claim 1, wherein the attaching step comprises:
attaching five electrodes to skin on the head of the patient.

3. The method of claim 2, wherein the attaching step comprises:
attaching five electrodes to skin on the head of the patient, one on the lower eyelid of the left eye, one on the lower eyelid of the right eye, one on the left temple, one on the right temple and one on the center of the forehead.

4. The method of claim 2, wherein the first displaying step comprises:
displaying a first set of stimuli patterns comprising a black-white bar grating on a display.

5. The method of claim 4, further comprising the steps of:
amplifying the first set of signals from the plurality of electrodes;
filtering the first set of signals from the plurality of electrodes;
digitizing the first set of signals from the plurality of electrodes;
averaging the first set of signals from the plurality of electrodes to produce an averaged first set of signals; and
noise canceling the averaged first set of signals.

6. The method of claim 1, further comprising the steps of:
displaying a second set of stimuli patterns to the patient;
receiving, in response to the second set of stimuli patterns, a second set of signals from the plurality of electrodes; and
averaging the first set of signals with the second set of signals to produce the source data set.

7. The method of claim 6, wherein the second displaying step comprises:
displaying a second set of stimuli patterns comprising a black-white bar grating on a display.

8. The method of claim 7, further comprising the steps of:
amplifying the second set of signals from the plurality of electrodes;
filtering the second set of signals from the plurality of electrodes;
digitizing the second set of signals from the plurality of electrodes;
averaging the second set of signals from the plurality of electrodes to produce an averaged second set of signals; and
noise canceling the averaged second set of signals.

9. The method of claim 8, wherein the averaging step comprises:
averaging the averaged first set of signals with the averaged second set of signals to produce a source data set.

10. The method of claim 9, wherein the comparing step comprises:
comparing the source data set with control data, wherein the control data comprises age-matched normal data and wherein a result of the comparing step is represented in standard deviations from an average of the control data.

11. A glaucoma screening system for detecting glaucoma in a patient, comprising:
a plurality of electrodes for attaching to the patient;
a display for displaying first and second sets of stimuli patterns viewed by the patient; and
a processor for receiving, in response to the first and second sets of stimuli patterns, first and second sets of signals from the plurality of electrodes, averaging the first set of signals with the second set of signals to produce a source data set and comparing the source data set with control data, wherein the processor is coupled to the plurality of electrodes and the display,
wherein a resultant value for determining whether the patient is afflicted with glaucoma is produced by the processor after comparing the source data set with the control data.

12. The system of claim 11, wherein the plurality of electrodes comprises:
five electrodes for attaching to skin on the head of the patient, one on the lower eyelid of the left eye, one on the lower eyelid of the right eye, one on the left temple, one on the right temple and one on the center of the forehead.

13. The system of claim 11, wherein the first set of stimuli patterns and the second set of stimuli patterns comprise a black-white bar grating.

14. The system of claim 11, wherein the processor comprises:
a first computer for receiving the first and second sets of signals from the plurality of electrodes, wherein the first computer is coupled to the plurality of electrodes and the display, the first computer including:
an amplifier for amplifying the first and second sets of signals;
a filter for filtering the first and second sets of signals;
a digitizer for digitizing the first and second sets of signals;
an averager for averaging the first and second sets of signals to produce an averaged first set of signals;
a noise reducer for noise canceling the averaged first and second sets of signals;
a module for averaging the averaged first set of signals with the averaged second set of signals to produce a source data set; and
a comparer for comparing the source data set with control data.

15. The system of claim 11, wherein the control data comprises age-matched normal data.

16. The system of claim 15, wherein the resultant value is:
represented in standard deviations from an average of the control data.

17. A computer readable medium comprising computer instructions for glaucoma screening in a patient, the computer instructions including instructions for:
displaying a first set of stimuli patterns to the patient;
receiving, in response to the first set of stimuli patterns, a first set of signals from a plurality of electrodes attached to the skin on the head of the patient;
displaying a second set of stimuli patterns to the patient;
receiving, in response to the second set of stimuli patterns, a second set of signals from the plurality of electrodes;
averaging the first set of signals with the second set of signals to produce a source data set;
comparing the source data set with control data; and
determining whether the patient is afflicted with glaucoma based on the instructions for comparing.

18. The computer readable medium of claim 17, wherein the first instructions for displaying comprise:
displaying a first set of stimuli patterns comprising a black-white bar grating on a display.

19. The computer readable medium of claim 18, further comprising the instructions of:
amplifying the first set of signals from the plurality of electrodes;
filtering the first set of signals from the plurality of electrodes;
digitizing the first set of signals from the plurality of electrodes;
averaging the first set of signals from the plurality of electrodes to produce an averaged first set of signals; and
noise canceling the averaged first set of signals.

20. The computer readable medium of claim 17, wherein the second instructions for displaying comprise:
displaying a second set of stimuli patterns comprising a black-white bar grating on a display.

21. The computer readable medium of claim 20, further comprising the instructions of:
amplifying the second set of signals from the plurality of electrodes;
filtering the second set of signals from the plurality of electrodes;
digitizing the second set of signals from the plurality of electrodes;
averaging the second set of signals from the plurality of electrodes to produce an averaged second set of signals; and
noise canceling the averaged second set of signals.

22. The computer readable medium of claim 21, wherein the instructions for averaging comprise:
averaging the averaged first set of signals with the averaged second set of signals to produce a source data set.

23. The computer readable medium of claim 22, wherein the instructions for comparing comprise:
comparing the source data set with control data, wherein the control data comprises age-matched normal data and wherein a result of the comparing step is represented in standard deviations from an average of the control data.
